(19) United States
(12) Patent Application Publication
(10) Pub. No.: US 2005/0139215 A1
(43) Pub. Date: Jun. 30, 2005

(54) Method for magnetic sinus therapy and magnetic nose pad apparatus

(76) Inventor: George Riach JR., Cypress, CA (US)

Correspondence Address: ORUM & ROTH, 53 W. JACKSON BLVD, CHICAGO, IL, 60604, US

(21) Appl. No.: 10/944,236

(22) Filed: Sep. 17, 2004

Related U.S. Application Data
(60) Provisional application No. 60/503,676, filed on Sep. 17, 2003.

(51) Int. Cl. A62B 7/10; A61M 16/00

(52) U.S. Cl. 128/206.11

(57) ABSTRACT

A magnetic sinus therapy applies at least one magnet oriented to direct a north magnetic field towards the nose/sinus area(s) of a user. The magnetic sinus therapy causes a reduction in blood aggregation of the blood in the nose/sinus area veins and capillaries. A reduction in blood aggregation improving circulation throughout the nose/sinus area as non-aggregated blood is better able to transfer oxygen, antibodies and immune system agents throughout the nose/sinus area. A method for sinus therapy may include the steps of taking a blood sample, exposing the nose/sinus area to a negative magnetic field for a selected time period and then taking another blood sample. The blood samples may then be compared with a microscope and or photomicrography to view the level of blood aggregation reduction that has resulted.

This application claims the benefit of U.S. Provisional Application 60,503,676 entitled Method for Magnetic Sinus Therapy and Magnetic Nose Pad Apparatus filed Sep. 17, 2003.

### BACKGROUND OF THE INVENTION

1. Field of the invention

The invention is related to magnets and magnetic fields. More specifically, the invention is related to a magnetic nose pad apparatus for use with a non-invasive magnetic method for sinus therapy.

2. Related Art

The nose and sinus system act as a filter for oxygen breathed into the body to sustain life. When the air carrying the oxygen is polluted with natural and or man made pollutants/irritants sinus conditions arise that cause considerable discomfort to the afflicted individuals. Toxic effects upon sinus tissue range from increased dryness or increased nasal discharge to an increased susceptibility to secondary infections. Attempts to chemically minimize the affects of these pollutant/irritants, for example with the use of anti-histamine drugs often leads to undesirable side effects, for example drowsiness.

Therapeutic magnetic devices have been used for a variety of localized disorders, including applicant's U.S. Pat. No. 5,389,981, titled “Eyeglasses having magnets attached thereto for improving the blood circulation of the eyes”, issued Feb. 14, 1995 and hereby incorporated by reference in the entirety.

As described in applicant's U.S. Pat. No. 5,389,981 patent, magnets have a north side (negative) and a south side (positive). The north side of a magnet provides beneficial effects when oriented to project a north magnetic field into living tissue. The beneficial effects including increasing the oxygen saturation of red blood cells exposed to the north magnetic field and a localized increase in blood circulation. The south side of a magnet generally provides non-beneficial effects when oriented to project into living tissue and can cause blood aggregation degeneration.

Therefore it is an object of the invention to provide a method for magnetic sinus therapy and an associated magnetic nose pad apparatus.

### SUMMARY OF THE INVENTION

A magnetic sinus therapy applies at least one magnet oriented to direct a north magnetic field towards the nose/sinus area(s) of a user. The magnetic sinus therapy causes a reduction in blood aggregation of the blood in the nose/sinus area veins and capillaries. A reduction in blood aggregation improving circulation throughout the nose/sinus area as non-aggregated blood is better able to transfer oxygen, antibodies and immune system agents throughout the nose/sinus area.

A method for sinus therapy may include the steps of taking a blood sample, exposing the nose/sinus area to a negative magnetic field for a selected time period and then taking another blood sample. The blood samples may then be compared with a microscope and or photomicrography to view the level of blood aggregation reduction that has resulted.

An apparatus for sinus therapy application may comprise one or more magnets removably adhered to a users nose or coupled to a retaining clip that is sized to fit over a user's nose. Alternatively, the magnet(s) may be added to existing or purpose built eyeglasses by incorporating one or more magnets into a solid nosepiece. Where, for example, with wire framed eyeglasses, silicon nose pads are present, the nose pads may have one or more magnets attached thereto.

### DESCRIPTION OF THE DRAWINGS

FIG. 1 is a front view of the nose area of a pair of wire frame eyeglasses with silicon type nose pads having magnets attached.

FIG. 2 is a front/side view of the nose area of a pair of wire frame eyeglasses with silicon type nose pads having magnets attached.

FIG. 3 is a front view of a nose clip with silicon nose pads having magnets attached.

FIG. 4 is a back view of the nose clip of FIG. 3.

FIG. 5 is a collection of wire frame eyeglasses with silicon pads having magnets attached.

FIG. 6 is a photograph of a person wearing eyeglasses with silicon pads having magnets attached.

FIG. 7 is an adhesive channel expanding device.

FIG. 8 is a person wearing a adhesive channel expanding device.

### DETAILED DESCRIPTION

Applicant's testing has shown that human blood reacts to exposure to south magnetic fields by increasing in aggregation level, i.e. increased clustering of the red blood cells towards each other. When human blood is exposed to north magnetic fields, blood aggregation levels decrease, increasing circulation and oxygen transfer.

Testing has shown that repeated exposure to magnetic fields oriented north, then south and back to north, etc., with blood samples taken for photomicrography after each exposure reveals that the blood aggregation may be increased or decreased solely via exposure to the respective magnetic field. Exposure time, for example 15 minutes, and field strength may be modified to achieve the desired level of blood aggregation reduction. Alternately, the body could be exposed to north magnetic fields as a means of preventing blood aggregation and its toxic effects upon the sinuses.

To localize the blood aggregation reduction effect of north magnetic fields to the sinus area as a method for sinus therapy, magnets may be located proximate the nose/sinus area oriented to project a north magnetic field into the nose/sinus area. For example, the magnet(s) 18 may be mounted on the nose pads 16 of most eyeglasses 10. The magnets may be used to prevent or reduce blood aggregation.

In the case of plastic eyeglasses 10 with integral molded solid nose areas, magnets oriented to project a north magnetic field into the nose/sinus area may be mounted to the solid nose area of the frame. Mounting may be perfected by, for example, adhering at least one magnet(s) 18 with a suitable adhesive, molding the magnet into the nose area at time of manufacture or by the creation of a suitable hole into which the magnet(s) 18 is/are press fit and or adhered.

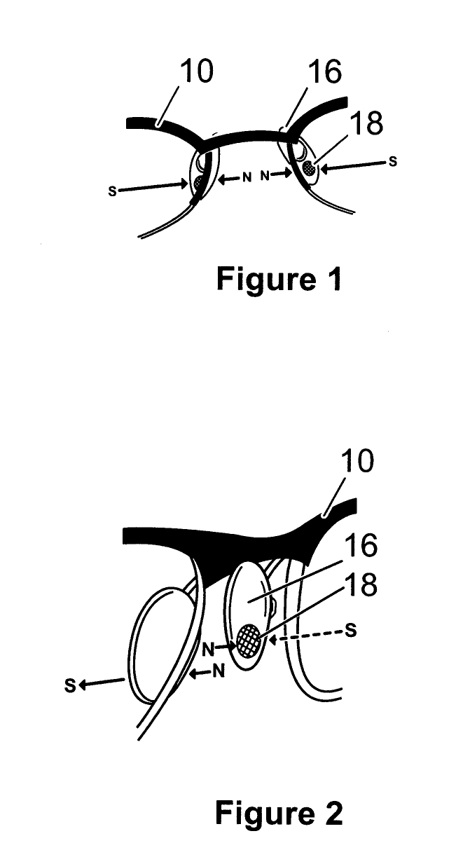

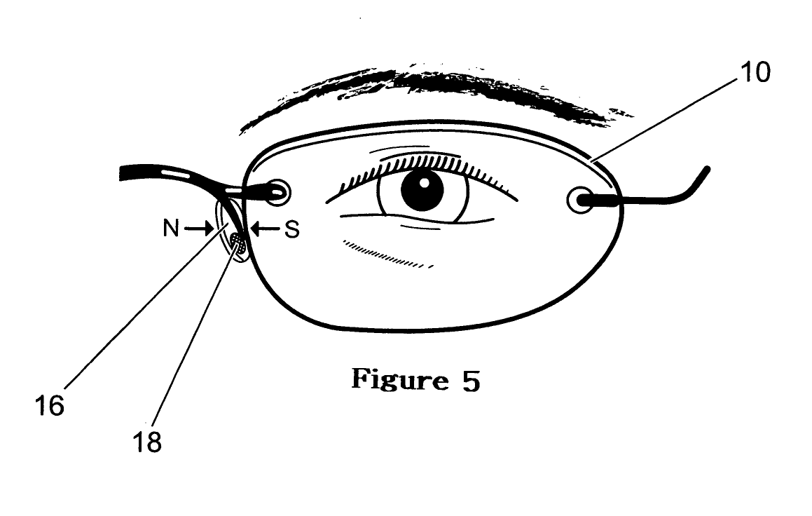

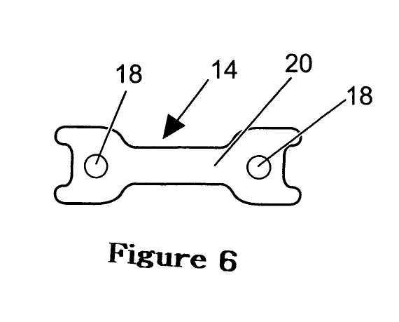

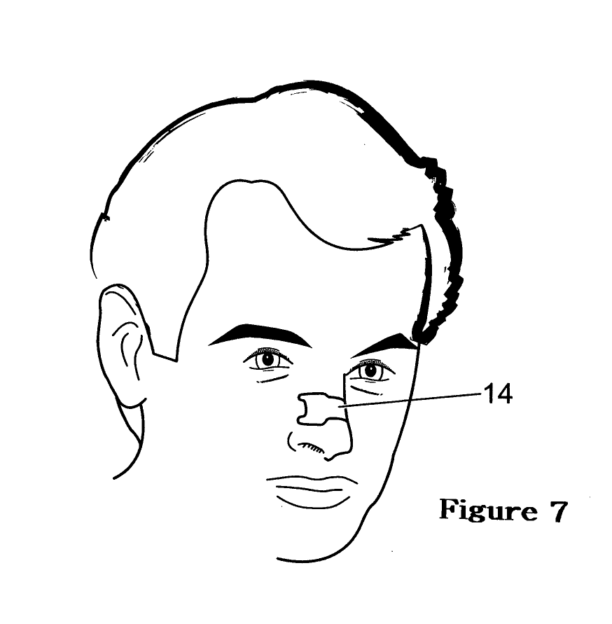

Wire frame glasses 10 typically use, for example, a pair of silicon pads 16 resting on the wearer's nose/sinus area supporting the wire frame and lenses. Magnets 18 oriented to project a north magnetic field into the nose/sinus area may be mounted on or within the silicon pads 16 as shown in FIGS. 1, 2, 5 and 6. Because these pads 16 are normally removable from the frame 10 for easy exchange, the invention may be added to an existing pair of eyeglasses 10 as a simple to install kit. The kit comprising one or more pads 16 with magnets 18 mounted thereto, oriented to project a north magnetic field towards the user, and a screw driver if necessary to remove existing nose pads and install the included nose pads 16 according to the invention.

The eyeglasses 10 can be manufactured with magnets 18 oriented to project a north magnetic field into the nose/sinus area or the magnets 18 can be added later such as with a do-it-yourself kit.

Magnets 18 oriented to project a north magnetic field into the nose/sinus area can be manufactured or added to safety glasses and goggles.

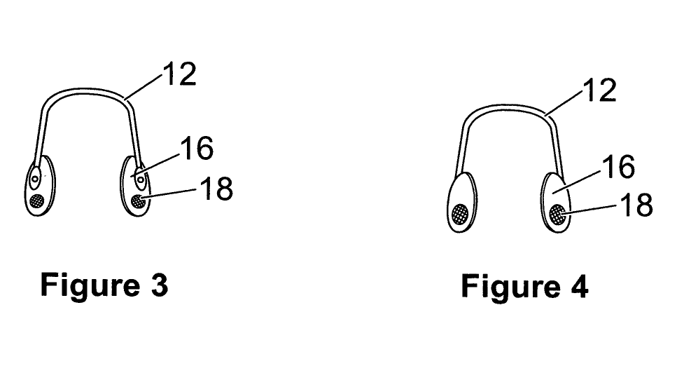

If a user does not wear glasses 10 or, for example, for use while sleeping without pillow interference, a magnetic nose clip 12 as shown, for example, in FIGS. 3 and 4 may be used. The nose clip 12 may use a for example metal or plastic clip 12 coupled with two nose pads 16. The nose pads containing magnets 18 as described herein, above.

In an alternative embodiment, the magnets 18 may be mounted on the form of adhesive 20 nose channel expanding devices 14 commonly used by athletes.

Alternatively, magnets 18 may be used with a releasable adhesive for adhering directly to the skin or be incorporated with an adhesive tape, for example surgical tape that is used to hold magnet(s) 18 in place over the nose/sinus area.

If there is a medical or other purpose for increasing blood aggregation, especially in the sinus area, the invention may be magnetically reversed. That is the fields may be oriented/selected so that the resulting user exposure is from the magnet's south magnetic field.

1. An apparatus for sinus therapy comprising:
a retaining device adapted to fit on a users nose, said retaining clip having at least one magnet oriented to project a magnetic north field into the users nose and/or sinus area.

2. The apparatus of claim 1 wherein the retaining device is a nosepiece of a pair of glasses.

3. The apparatus of claim 2 further comprising at least two magnets wherein one magnet is located on each side of the nosepiece.

4. The apparatus of claim 1, wherein the device is a unshaped clip comprising a pair of nose pads and one pad is located on each leg of the clip and there is a magnet on each nose pad.

5. The apparatus of claim 1 wherein the retaining device is an adhesive strip.

6. The apparatus of claim 5 wherein the device is disposable.

7. A method of sinus therapy comprising the steps of:
directing a magnetic north field into the nose and/or sinus area, after a specified period of time removing the magnetic field.

8. The method of claim 6 comprising the additional step:
donning a retaining device having one or more magnets said retaining device adapted to fit a user's nose.

9. The method of claim 6 comprising the additional step of applying one or more magnets to the retaining device.
